(19) United States
(12) Patent Application Publication
(10) Pub. No.: US 2012/0021034 A1
(43) Pub. Date: Jan. 26, 2012

(54) STRUCTURED SILVER-MESOPOROUS SILICA NANOPARTICLES HAVING ANTIMICROBIAL ACTIVITY

(75) Inventors: Jeffrey I. Zink, Sherman Oaks, CA (US); Monty Liong, Los Angeles, CA (US); Bryan D. France, Los Angeles, CA (US); Kenneth A. Bradley, Los Angeles, CA (US)

(73) Assignee: The Regents of the University of California, Oakland, CA (US)

(21) Appl. No.: 13/140,714

(22) PCT Filed: Dec. 18, 2009
(86) PCT No.: PCT/US09/68816

§ 371 Date: Jun. 17, 2011

Related U.S. Application Data
(60) Provisional application No. 61/139,310, filed on Dec. 19, 2008.

(51) Int. Cl. A01N 25/26; A01P 1/00; A01N 59/16; B82Y 5/00

(52) U.S. Cl. 424/421; 424/618; 424/178.1; 977/773; 977/904

(57) ABSTRACT

A submicron structure having a silica body defining a plurality of pores, said silica body further defining an outer surface between pore openings of said plurality of pores; and having at least one silver nanocrystal within said silica body are described. Antimicrobial compositions comprising the submicron structure, and methods of killing or inhibiting growth of microbes using the submicron structure are described.

### CROSS-REFERENCE OF RELATED APPLICATION

This application claims priority to U.S. Provisional Application No. 61/139,310 filed Dec. 19, 2008, the entire contents of which are hereby incorporated by reference.

This invention was made with Government support of Grant No. CHE 0809384, awarded by the National Science Foundation and Grant No. AI057870, awarded by the National Institutes of Health. The Government has certain rights in this invention.

### BACKGROUND

1. Field of Invention

The current invention relates to silver-core, mesoporous silica nanoparticles, and more particularly to silver-core, mesoporous silica nanoparticles having antimicrobial activity.

2. Discussion of Related Art

MCM-41 type mesoporous silicate particles have attracted significant amounts of research interest due to the ordered porous structure of the materials, their facile synthetic methods, and broad range of applications (Beck et al., J. Am. Chem. Soc., 1992, vol. 114, p. 10834; Ying et al., Angew. Chem., Int. Ed, 1999, vol. 38, p. 56; Saha et al., Adv. Funct. Mater. 2007, vol. 17, p. 685; Angelos et al., Adv. Funct. Mater. 2007, vol. 17, p. 2261). Further developments in the synthesis and modification of nano-sized mesoporous silica materials have created new possibilities for biomedical applications (Cai et al., Chem. Mater., 2001, vol. 13, p. 258; Lin et al., Chem. Mater, 2005, 17, 4570; Möller et al., Adv. Funct. Mater. 2007, 17, 605; Lu et al., Small 2008, 4, 421) As opposed to nonporous silica nanoparticles, both the surface and the pore interior of mesostructured nanoparticles can be modified with functional groups such that they become compatible in various solutions and are able to store different types of molecules (Stein et al., Adv. Mater., 2000, vol. 12, p. 1403; Vallet-Regi et al., Angew. Chem., Int. Ed., 2007, vol. 46, p. 7548; Kobler et al., ACS Nano, 2008, vol. 2, p. 791; Nguyen et al., J. Am. Chem. Soc., 2007, vol. 129, p. 626; Nguyen et al., Org. Lett., 2006, vol. 8, p. 3363; Minoofar et al., J. Am, Chem. Soc., 2002, vol. 124, p. 14388; Minoofar et al., J. Am. Chem. Soc. 2005, vol. 127, p. 2656). These nanomaterials have been well demonstrated for their biocompatibility (Barbe et al., Adv. Mater., 2004, vol. 16, p. 1959; Taylor et al., J. Am. Chem. Soc., 2008, vol. 130, p. 2154), and in their utilization as fluorescent markers for cells (Wu et al., ChemBioChem, 2008, vol. 9, p. 53), gene-transfection agents (Radu et al., J. Am. Chem. Soc., 2004, vol. 126, p. 13216), and delivery vehicles for proteins and anticancer drugs (Slowing et al., J. Am. Chem. Soc. 2007, vol. 129, p. 8845; Lu et al., Small, 2007, vol. 3, p. 1341).

Silver has been known to act antimicrobially as an agent in and on the body of humans as well as other animals, and to be relatively non-toxic to mammalian cells when used in the minute quantities needed to be antimicrobially effective. The most effective form of silver for antimicrobial use is as ions in solution. Silver ions have been shown in the past to have antibacterial, antiviral and antifungal qualities, and to contribute directly to the regeneration of tissue. While the exact method by which silver ions perform these functions is not known, it is believed that they may (1) disrupt the respiratory functions, or (2) disrupt membrane functionality of single-celled microorganisms, or (3) link to the cell's DNA and disrupt cell functions. It is not conventionally understood why silver ions appear to some to be effective at regenerating tissue, which apparently involves more than acting as an antimicrobial agent.

Antibiotic-resistant microorganisms cause numerous problems and infections in various facilities. Although the antimicrobial activity of silver nanoparticles is well known and has proven effective against antibiotic-resistant strains, the materials are typically prone to aggregation and incompatible in a biological environment. There thus remains a need for improved antimicrobial materials that contain silver.

### SUMMARY

Embodiments of the invention include a submicron structure having a silver core and a silica body formed around said silver core. The silica body defines a plurality of pores, and an outer surface between pore openings of said plurality of pores. The submicron structure has a maximum dimension less than one micron (μm). In some embodiments, the silver core is a silver nanocrystal. In some embodiments, the silica body is mesoporous. In some embodiments, the surface(s) of the silica bodies are modified.

Other embodiments of the invention include compositions having a plurality of submicron structures described above. In some embodiments, the compositions further include a suspending liquid, or a fiber or polymer material. The compositions are useful as antimicrobial materials.

Other embodiments of the invention include methods of killing or inhibiting the growth of microbes by contacting the microbes with the submicron structures described above. In some embodiments, the microbe is a bacteria.

### BRIEF DESCRIPTION OF THE DRAWINGS

Further objectives and advantages will become apparent from a consideration of the description, drawings, and examples.

FIG. 1A shows scanning electron microscope and FIG. 1B shows transmission images of the silver nanocrystals encapsulated in mesoporous silica nanoparticles. The silver (black spheres) in the larger silica (light spheres) is most clearly seen in FIG. 1B.

FIG. 2A shows Petri dishes with LB-agar inoculated with Bacillus anthracis and FIG. 2B shows Petri dishes with LB-agar inoculated with Eschericia. coli showing variable numbers of colonies when supplemented with different amounts of nanoparticles.

FIG. 3 shows bacterial growth curve in LB Lennox liquid media. Different concentrations of silver encapsulated mesoporous silica nanoparticles (NP) or PEI-coated nanoparticles (PEI-NP) were added to the B. anthracis (FIGS. 3A and 3C) and E. coli (FIGS. 3B and 3D) culture. The growth of the bacteria was monitored by measuring the OD₆₀₀. Each data point represents a minimum of three independent experiments shown with standard error of the mean.

FIG. 4 shows Fluorescence microscopy images of B. anthracis (FIGS. 4A and 4B) and E. coli (FIGS. 4C and 4D) treated with either unmodified nanoparticles (FIGS. 4B and 4D) or PEI-coated nanoparticles (PEI-NPs) (FIGS. 4A and 4C) Rhodamine B-labeled Ag@MESs.

FIG. 5 shows UV-Vis extinction spectra of the Rhodamine α-labeled Ag@MESs suspended in LB Lennox media (FIG. 5A) and deionized water (FIG. 5B). The surface plasmon peak of silver (425 nm) decreased over time in the culture media, but remained relatively unchanged in water.

FIG. 6 shows fluorescence microscopy images of E. coli (FIG. 6A) and B. anthracis (FIG. 6B) treated with Rhodamine B-labeled mesoporous silica nanoparticles.

FIG. 7 shows Transmission electron microscope images of oleylamine-capped silver nanocrystals (FIG. 7A) and Ag@MESs at higher magnification (FIG. 7B). X-ray diffraction pattern of Ag@MESs shows an interplanar spacing of d(100)=4.2 nm (FIG. 7C).

FIG. 8 shows Extinction spectrum of the oleylamine-capped silver nanocrystals (Ag NC, in chloroform), CTAB-stabilized silver nanocrystals (CTAB-Ag NC, in water), and silver encapsulated mesoporous silica nanoparticles (Ag@MES, in water).

FIG. 9 shows growth curve of B. anthracis (FIG. 9A) and E. coli (FIG. 9B) in the presence of either PEI-coated (PEI-MSN) or unmodified mesoporous silica nanoparticles (MSN)

FIG. 10 shows the effect of silver nitrate on the growth of B. anthracis (FIG. 10A) and E. coli (FIG. 10B).

FIG. 11 shows transmission electron microscope images of oleate-capped iron oxide nanocrystals (FIG. 11A) and mixed iron oxide and silver encapsulated in mesoporous silica nanoparticles (FIG. 11B).

FIG. 12 shows a suspension of E. coli stained with Hoechst 33342 that was incubated with the PEI-coated iron oxide and silver encapsulated mesoporous silica nanoparticles (10 mg/mL). The binding of particles to the bacteria allow the cells to be collected from solution using the neodymium magnet.

### DETAILED DESCRIPTION

Some embodiments of the current invention are discussed in detail below. In describing embodiments, specific terminology is employed for the sake of clarity. However, the invention is not intended to be limited to the specific terminology so selected. A person skilled in the relevant art will recognize that other equivalent components can be employed and other methods developed without departing from the broad concepts of the current invention. All references cited anywhere in this specification are incorporated by reference as if each had been individually incorporated.

Embodiments of the invention include a submicron structure, comprising a silver core; and a silica body formed around said silver core. The silica body defines a plurality of pores, and an outer surface between pore openings of said plurality of pores. The submicron structure has a maximum dimension less than one micron (m). In some embodiments, the silver core can be one or more silver nanocrystals.

The submicron structure includes a silica body that defines a plurality of pores therein. For example, the silica body can be a mesoporous silica nanoparticle. The fact that we refer to the body as a silica body does not preclude materials other than silica from also being incorporated within the silica body. In some embodiments, the silica body may be substantially spherical with a plurality of pore openings through the surface providing access to the pores. However, the silica body can have shapes other than substantially spherical shapes in other embodiments of the current invention. Generally, the silica body defines an outer surface between the pore openings, as well as side walls within the pores. The pores can extend through the silica body to another pore opening, or can extend only partially through the silica body such that it has a bottom surface of the pore defined by the silica body.

The robust silica shell protects the silver core from aggregation and fast dissolution, and provides support for surface modification with functional groups. The pores of the silica coating allow small molecules (for example, solvents, amino acids, peptides) and ions to diffuse into the nanoparticles and interact with the silver nanocrystals. This process, in turn, leads to the release of silver ions and an antimicrobial effect. Surface modification of the silica shell can provide dispersibility in both polar and nonpolar solvents. Additionally, various functional groups can be introduced onto the silica surface in order to conjugate the nanoparticles with other molecules or substrates.

In some embodiments, the silica body is mesoporous. In other embodiments, the silica body is microporous. As used herein, “mesoporous” means having pores with a diameter between 2 nm and 50 nm, while “microporous” means having pores with a diameter smaller than 2 nm. In general, the pores may be of any size capable of allowing the silver nanocrystal within the silica body to interact with the environment outside the silica body. The pores allow small molecules, for example, peptides or ions, to diffuse into the nanoparticles and interact with the silver core in the silica body. The pores also allow silver ions from the silver core to diffuse out of the silica body. In some embodiments, the pores are substantially cylindrical. Some embodiments of the invention include nanoparticles having pore diameters between about 1 nm and about 10 nm in diameter. Other embodiments include nanoparticles having pore diameters between about 1 nm and about 5 nm. Other embodiments include particles having pore diameters less than 2.5 nm. In other embodiments, the pore diameters are between 1.5 and 2.5 nm.

The submicron structures according to some embodiments of the current invention may be referred to as nanoparticles. The term nanoparticles as used herein is intended the include particles as large as 1000 nm. In general, particles larger than 300 nm become ineffective in entering living cells. Particles greater than 300 nm in diameter may be effective as antimicrobials, however, because they interact with the surface of the microbe, rather than entering the microbial cell. In some embodiments, colloidal suspensions may be formed using a plurality of submicron structures according to some embodiments of the invention. In that case, larger particles can tend to settle rather than remaining suspended in Brownian motion. Some embodiments include nanoparticles having a maximum dimension between about 50 nm and about 1000 nm. Other embodiments include nanoparticles having a maximum dimension between about 100 nm and about 500 nm. Other embodiments include nanoparticles having a maximum dimension between about 100 nm and about 200 nm.

In some embodiments, the silver nanocrystal has a maximum dimension of less than about 50 nm. In some embodiments, the silver nanocrystal has a maximum dimension of less than about 20 nm. In some embodiments, the silver nanocrystal has a maximum dimension between about 1 nm and about 50 nm. In other embodiments, the silver nanocrystal has a maximum dimension between about 1 nm and about 20 nm. In some embodiments, the silver nanocrystal is larger than the pore size of the nanoparticle.

In some embodiments, the submicron structure described above further includes a stopper assembly attached to the silica body. The stopper assembly has a blocking unit arranged proximate at least one pore and has a structure suitable to substantially prevent material from entering or being released when the blocking unit is arranged in a blocking configuration. The stopper assembly is responsive to the presence of a predetermined stimulus such that the blocking unit is released in the presence of the predetermined stimulus to allow material to enter or be released. The predetermined stimulus is a predetermined catalytic activity that is suitable to cleave, hydrolyze, oxidize, or reduce a portion of the stopper assembly. Examples of stopper assemblies are described, for example, in International Application No. PCT/US2009/031891, filed Jan. 23, 2009, now published as WO 2009/094580 and incorporated herein by reference in its entirety.

In some embodiments the stopper assembly can include a thread onto which the blocking unit can be threaded. The thread has a longitudinal length that is long relative to a transverse length and is suitable to be attached at one longitudinal end to the silica body. The stopper assembly can also have a stopper attached to a second longitudinal end of the thread in some embodiments. The stopper can be selected among a wide range of possible stoppers based on the type of environment

For example, according to some embodiments, a synthetic strategy can involve the use of a snap-top “precursor”. The assembly of the snap-top precursors can be performed step-wise from the silica nanoparticle surfaces outward. For instance, the silica nanoparticles are treated with aminopropyltriethoxysilane (APTES) to achieve an amine-modified nanoparticle surface. An azide terminated tri(ethylene)glycol thread is attached to the amine-modified nanoparticles. The precursor is completed through the addition of α-cyclodextrin as the blocking unit at 5° C., which complexes with the threads at the low temperature. The precursor can enable the preparation of many different systems based on a common general structure in which different stoppers can be attached depending on the specific desired application. For example, stoppers may be selected that respond to enzymes (for example, ester linked or peptide linked), pH (for example, vinyl ether linked), and redox (for example disulfide linked) stimulation. However, the broad concepts of the current invention are not limited to only these specific examples. There are a wide range of possible stoppers that may be selected according to the particular application.

Other embodiments include submicron structures further including an impeller attached to the silica body. Silica bodies modified by impellers are described, for example in International Application No. PCT/US2009/031871, filed Jan. 23, 2009, published as WO 2009/094568, the contents of which are incorporated herein in their entirety. The term “impeller” as used herein is intended to have a broad meaning to include structures which can be caused to move and which can in turn cause molecules located proximate the impeller to move in response to the motion of the impeller.

In operation, the impellers are driven by an energy transfer process. The energy transfer process can be, but is not limited to, absorption and/or emission of electromagnetic energy. For example, illuminating with light at an appropriate wavelength can cause the plurality of impellers to wag back and forth between two molecular shapes. The motion of the plurality of impellers causes motion of molecules (for example, peptides, proteins, ions, drugs or antibiotics) of interest into and/or out of the silica body. On the other hand, in the absence of excitation energy, the plurality of impellers can remain substantially static, at least for time periods long enough for the desired application, to act as impediments to block molecules from exiting and/or entering the storage chamber.

The impellers can be, but are not limited to, azobenzenes according to some embodiments of the current invention. For example, the azobenzenes can include the following: 1) One phenyl ring derivatized with a functional group that enables attachment directly to the silica surface or to a modified silica surface as described later. The list of suitable functional groups contains but is not limited to: alcohols, (—ROH), anilinium amines (—NH₂) primary amines (—RNH₂), secondary amines (—R₁R₂NH), azides (N₃), alkynes (RC≡CH), isocyanates (—RNCO), isothiocyanates (—RNCS), acid halides (RCOX), alkyl halides (RX) and succinimidyl esters. 2) other functional groups on the other phenyl ring (which is the moving end of the machine). The list of these functional groups includes but is not limited to: —H (here the phenyl ring is underivatized), esters (—OR), primary and secondary amines, alkyl group, polycyclic aromatics, and various generations of dendrimers. The bulkiness of these functional groups can be designed for specific systems. For example, large dendritic functionalities might be required when very large pore openings or very small guest molecules are employed.

When illuminated or irradiated with light of a particular wavelength, the azobenzene undergoes photoisomerization, causing the second phenyl group to move.

In other embodiments, impellers are based on redox of copper complexes. The copper complexes can include bifunctional bidentate stators that contain diphosphine and/or diimine bidentate metal chelators on one end of the stator, while at the other end functionalities such as alkoxysilanes (for immobilization on silica and silicon substrates) and thiols (for immobilization on gold substrates) are present. The copper complexes can contain a rotator that is a rigid bidentate diimine metal chelator, which rotates and changes the shape of the overall molecule upon redox or photons. These copper complexes exist in two oxidation states, each of which corresponds to a specific shape. Copper (I) is tetrahedral while copper (II) is square planar. The different oxidation states, and hence different shapes that are caused by a 90° rotation of the rotator, can be generated in three ways: Reduction and oxidation (1) using electrodes and an electric current (2) by use of chemical reducing and oxidizing agents, and (3) by the photo-excitation of light of the appropriate wavelength.

Some embodiments include submicron structure further including a valve assembly attached to the silica body. Porous nanoparticles having valves are described, for example, in International Application No. PCT/US2009/032451, filed Jan. 29, 2009, published as WO 2009/097439, the contents of which are incorporated herein in their entirety. In some embodiments, the valve assembly is operable in an aqueous environment. The valve assembly has a valve arranged proximate the at least one pore and has a structure suitable to substantially prevent material from entering or being released while the valve is arranged in a blocking configuration. The valve assembly is responsive to a change in pH such that the valve moves in the presence of the change in pH to allow the material to enter or be released from the silica body.

According to some embodiments of the current invention, the pH-responsive valve assembly relies on the ion-dipole interaction between cucurbit[6]uril (CB[6]) and bisammonium stalks, and that can operate in water. CB[6], a pumpkin-shaped polymacrocycle with D₆ₕ symmetry consisting of six glycouril units strapped together by pairs of bridging methylene groups between nitrogen atoms has received considerable attention because of its highly distinctive range of physical and chemical properties. Of particular interest in the field of supramolecular chemistry is the ability of CB[6] to form inclusion complexes with a variety of polymethylene derivatives, especially diaminoalkanes: the stabilities of these 1:1 complexes are highly pH-dependent. The pH-dependent complexation-decomplexation behavior of CB[6] with diaminoalkanes has enabled the preparation of dynamic supramolecular entities which can be controlled by pH. In some embodiments, [2]pseudorotaxanes having bisammonium stalks and CB[6] rings, may be constructed on the surface of the mesoporous silica nanoparticles, and the pH-dependent binding of CB[6] with the bisammonium stalks is exploited to control the entry or release of molecules from the silica nanoparticles. At neutral and acidic pH values, the CB[6] rings encircle the bisammonium stalks tightly, blocking the nanopores efficiently when employing suitable lengths of tethers. Deprotonation of the stalks upon addition of base results in spontaneous dethreading of the CB[6] rings and unblocking of the pores.

In some embodiments, the surface of the submicron structure or nanoparticle is unmodified. As used herein, an “unmodified” nanoparticle has had no other functional groups added to the surface after formation of the nanoparticle. Unmodified nanoparticles have an anionic charge due to free silyl hydroxide moieties present on the surface.

Other embodiments include submicron structure as described above, which further include a surface modification. As used herein, “surface modification” means attaching or appending molecules or other materials to the surface of the silica body. The surface modification may be covalent, electrostatic or a combination of both. For example, the surface may include a covalent surface modification and an electrostatic surface modification on the same nanoparticle. In some embodiments, the surface modification may be further derivatized, for instance, by further covalent or electrostatic bonds. Surface modifications, as described herein, may be used on any silica body having an unreacted silica surface, including nanodevices having stoppers, impellers or valves, as described above.

In some embodiments, the surface modification comprises a plurality of anionic or electrostatic molecules attached to an outer surface of said silica body, wherein the anionic or electrostatic molecules provide hydrophilicity or aqueous dispersability to the nanoparticle and are suitable to provide repulsion between other similar submicron structures. Anionic surface modified nanopartices are described, for example, in International Application No. PCT/US2008/013476, filed Dec. 8, 2008, published as WO 2009/078924, the contents of which are incorporated herein by reference in its entirety.

In some embodiments, the plurality of anionic molecules include at least one phosphonate moiety. In some embodiments, the plurality of anionic molecules are trihydroxysilylpropyl methylphosphonate. Trihydroxysilylpropyl methyl phosphonate surface modifications are prepared, for example, by treating the silica body with trihydroxysilyl propyl methylphosphonate.

In some embodiments, the surface modification is covalently bonded to the surface of the silica body. In other words, the surface modification has a functional group covalently bonded to the surface. As used herein, the “functional group” defines a chemical moiety linked to the surface of the nanoparticle, either directly, or via a linker. In some embodiments, the functional group is an amine, sulfhydryl, disulfide, carboxylic acid, epoxide, halide (i.e. fluorine, chorine, bromine, or iodine), azide, alkyne, or hydrophobic moiety. In some embodiments, the functional group is an amide. In some embodiments, the functional group may be further bonded, covalently or electrostatically to a further compound.

In general, any reaction capable of reacting with the silyl hydroxide surface of the silica body may be used to covalently modify the surface. For example, the surface of the silica body may be treated with a trialkoxysilyl compound. The trialkoxysilyl compound reacts with the silyl hydroxide surface of the silica body, forming covalent silicon-oxygen bonds. Trialkoxysilyl compounds bearing various functional groups may be used to modify the surface of the nanoparticle.

In some embodiments, the covalent surface modification comprises an amine, sulfhydryl, disulfide, carboxylic acid, epoxide or hydrophobic organic moiety. As discussed above, various functional groups may be present on the surface modification, depending on the reagents used to modify the surface. In some embodiments, the functional group (i.e. amine, sulfhydryl, disulfide, carboxylic acid, epoxide, halide, azide, alkyne, or hydrophobic organic moiety) may be separated from the silica surface by a linker. In some embodiments, the functional group is covalently bonded to the silica surface via a C₁ to C₁₂ alkyl linker. In other words, a C₁ to C₁₂ alkyl group is present between the atom covalently bonded to the surface and the functional group (i.e. amine, sulfhydryl, disulfide, carboxylic acid, epoxide or hydrophobic organic moiety). In other embodiments, the functional group is covalently bonded to the silica surface via a C₁ to C₆ alkyl linker. Nanoparticles bearing a surface modification are called surface-modified nanoparticles.

As used herein a C₁ to C₁₂ alkyl chain includes linear, branched and cyclic structures having 1 to 12 carbon atoms, and hybrids thereof, such as cycloalkylalkyl. Examples of alkyl chains include methylene (CH₂), ethylene (CH₂CH₂), propylene (CH₂CH₂CH₂), and so forth.

A used herein, surface modifications having an amine (also known as amine modified nanoparticles) will have at least one primary (—NH₂), secondary (—NHR), tertiary (—NR₂) or quaternary amine. An amine-modified surface may be charged or uncharged, depending on the amine and pH. Amine modifications may be prepared, for example, by treating the silica body surface with an amine bearing trialkoxysilane compound, such as aminopropyltriethoxysilane, 3-(2-aminoethylamino)propyl-trimethoxysilane, or 3-trimethoxysilylpropyl ethylenediamine.

As used herein, surface modifications having a sulfhydryl (or thiol) group will have at least one —SH moiety. Such a modification may be prepared, for example, by treating the surface of the nanoparticle with a sulfyhdryl bearing trialkoxysilane compound, such as 3-mercaptopropyltriethoxysilane.

As used herein, surface modifications having a disulfide group will have at least one —S—S— moiety. Such a modification may be prepared, for example, by treating the surface of the nanoparticle with a disulfide bearing trialkoxysilane compound, or by treating a sulfhydryl modified surface with 2,2′-dithiodipyridine or other disulfide.

Surface modifications having a carboxylic acid group will have at least one —CO₂H, or salt thereof. Such a modification may be prepared, for example, by treating the surface with a carboxylic acid bearing trialkoxysilane compound, or by treating the surface with a trialkoxysilane compound bearing a functional group that may be converted chemically into a carboxylic acid. For example, the surface may be treated with 3-cyanopropyltriethoxysilane, followed by hydrolysis with sulfuric acid.

Surface modifications having an epoxide will have at least one epoxide present on the surface of the nanoparticle. Such a modification may be prepared, for example, by treating the surface with an epoxide bearing trialkoxysilane compound, such as glysidoxypropyltriethoxysilane.

Surface modifications having a hydrophobic moiety will have at least moiety intended to reduce the solubility in water, or increase the solubility in organic solvents. Examples of hydrophobic moieties include long chain alkyl groups, fatty acid esters, and aromatic rings.

Any of the covalent surface modifications described above may be further derivatized, for example, by further covalent or electrostatic bonds. In some embodiments, the surface modification is further covalently bonded to another compound, such as a light-emitting molecule, peptide, protein, nucleic acid, sugar, oligosaccharide, or polysaccharide. Light emitting molecules include compounds which emit light by either fluorescence or phosphorescence. Light emitting molecules include dyes, such as fluorescent dyes. Examples of light emitting molecules include fluorescent dyes such as fluorescein, and rhodamine B. Light emitting molecules may be covalently bonded to the surface modified silica body by any useable method. For example, amine-modified nanoparticles having a free NH₂ group may be reacted with fluorescent dyes bearing amine-reactive groups such as isocyanates, isothiocyanates, and activated esters, such as N-hydroxysuccinimide (NHS) esters. Examples of fluorescent dyes bearing amine reactive groups include, for example, fluoresceine isothiocyanate, N-hydroxysuccinimide-fluorescein, rhodamine B isothiocyanate, or tetramethylrhodamine B isothiocyanate. Other dyes will be apparent to those of skill in the art. Nanoparticles bearing light-emitting molecules may be used, for example, for fluorescence imaging, for instance when the nanoparticles interact with the surface of a microbe.

In some embodiments, the surface modification is further bonded to a peptide or protein. Peptides include polypeptides having at least 2 amino acids. Various amino acid residues on peptides or proteins may form a covalent bond with surface-modified nanoparticles. For example, carboxylic acid residues (from aspartic acid and glutamic acid) may react with amine-modified nanoparticles bearing a free NH₂ group. Likewise, amine residues on proteins (i.e. from lysine) may react with carboxylic acid bearing surface modifications or with epoxide bearing surface modifications. Sulfhydryl surface modifications may react with disulfide bonds (e.g. from cystine residues) in the protein via thiol exchange. Disulfide surface modifications, such as 2-thiopyridine disulfides may react with free thiols (e.g. from cysteine residues) in the protein to form a covalent bond with the protein. Other suitable methods for conjugating the proteins to the surface-modified nanoparticles will be evident to those of skill in the art.

In some embodiments, the protein, peptide, oligonucleotide, sugar, oligosaccharide, or polysaccharide is covalently attached to the surface modifying group via a linker. Various bifunctional crosslinkers are known to those in the art for covalently bonding to proteins, any of which may be used to covalently link a surface modified nanoparticle to a protein. For example, heterodifunctional crosslinkers such as succinimidyl-4-[N-maleimidomethyl]cyclohexane-1-carboxylate (SMCC) and melaimidobutyryloxysuccinimide ester (GMBS) may be used to react with amine-modified nanoparticles (via the succinimide esters), and then form a covalent bond with a free thiol in the protein (via the maleimide). Other crosslinkers, such as succinimidyl 3-(2-pyridyldithio)-propionate (SPDP) may react with amine-modified nanoparticles (via the succinimide ester), and form a covalent bond with a free thiol in the protein via thiol exchange. Other difunctional crosslinkers include suberic acid bis(N-hydrosuccinimide ester), which can react with amine-modified nanoparticles, and free amines on the protein (e.g. from lysine residues). Other bifunctional and heterobifunctional crosslinkers useable with various surface modifications will be evident to those of skill in the art.

In some embodiments, the surface modification is electrostatically bonded to the surface. As used herein “electrostatically bonded” means bonded based on the attraction of opposite charges. As described previously, an unmodified nanoparticle has a negative charge, due to the presence of free silyl hydroxide residues on the surface of the nanoparticle. The particle may also bear a surface modification having a negative charge, such that the overall charge of the surface is negative. The surface may be modified with material bearing a positive charge, which will bind to the surface electrostatically.

In some embodiments, the surface modifying material is a polymer, protein, peptides, nucleic acids, sugars, oligosaccharides, polysaccharides, or combination thereof.

In some embodiments, the surface modifying material is a cationic polymer, such as, for example, poly(ethyleneimine) (PEI), poly(allylamine) or poly(diallyldimethylammonium chloride). Other cationic polymers will be apparent to those of skill in the art. Cationic polymer modified nanoparticles have a positive charge.

In other embodiments, the surface modifying material is a protein that binds to the surface electrostatically. A protein having a net positive charge will bind electrostatically to unmodified nanoparticles or surface modified nanoparticles bearing a negative charge. For example, proteins such as Bovine Serum Albumin (BSA) and protein solutions such as Fetal Bovine Serum (FBS) bind electrostatically to unmodified nanoparticles.

A protein having a net negative charge will bind electrostatically to modified nanoparticles having a positive charge, such as amine-modified nanoparticles, or nanoparticles modified by cationic polymers. For example, proteins such as the anthrax toxin receptor (ANTXR) bind electrostatically to nanoparticles modified by PEI.

In some embodiments, the surface modification is a combination of a polymer and a protein.

Surface-modified nanoparticles bearing a protein are also called protein-modified nanoparticles. The protein may be bonded covalently (directly to the surface modification or via a linker) or may be electrostatically bonded to the modified or unmodified nanoparticles as discussed above. The protein may be a targeting protein or an antibody. A “targeting protein” as used herein, means a protein which binds to a particular surface feature of a microbe or substrate of interest. For instance, the anthrax toxin receptor (ANTXR) discussed previously, binds to the surface of Bacillus anthracis spores. Antibodies and peptides are also used to bind to particular surface features of microbes, and may be used to modify the nanoparticles of the invention. Protein-modified nanoparticles may be used to selectively target specific microbes, by interacting specifically or selectively to a microbe of interest.

Other embodiments include submicron structures having both a silver core and a second core structure in the nanoparticle. In some embodiments, the submicron structure has both a silver nanocrystal and a non-silver nanocrystal. In some embodiments, the non-silver nanocrystal is a superparamagnetic nanocrystal, such as, for example, an iron oxide nanocrystal. A superparamagnetic nanocrystal core makes the particles visible using magnetic resonance imaging (MRI). The nanoparticles may be used as MRI contrast agents having an antimicrobial property. The superparamagnetic nanocrystal in the core of the nanoparticle also allows the particles to be manipulated or collected by a magnetic field, for example. This feature is useful, for example, in antimicrobial cleaning products. The nanoparticles have a silver nanocrystal to kill microbes or inhibit their growth, and a superparamagnetic nanocrystal to be manipulated by a magnetic field. The nanoparticles may be surface modified to bind to certain types of microbes (for example, Gram negative or Gram positive bacteria). Any microbes bound to the surface of the nanoparticles may also be collected or manipulated using a magnetic field. In this way, potentially harmful microbes may be concentrated or collected in a particular location, thereby reducing or the amount of cleaning product which must be disposed of according to biohazard protocols.

In other embodiments, the non-silver nanocrystal is a gold nanocrystal. In some non-limiting embodiments, gold nanocrystals produce an antimicrobial effect through light irradiation. For instance, light irradiation induces radical oxygen species (ROS) production by gold. In other cases, when illuminated with light, the temperature of gold particles increases, killing cells (i.e. by hyperthermia). Nanoparticles having both silver and gold nanocrystals will have the combined antimicrobial properties of the silver and gold nanocrystals.

The robust mesoporous silica shell protects the core silver nanocrystals from aggregation and fast dissolution, and provides support for surface modification with functional groups. The pores of the silica coating allow small molecules (for example, peptides) and ions to diffuse into the nanoparticles and interact with the silver nanocrystals. This process will in turn lead to the release of silver ions and the antimicrobial effect. Surface modification of the silica shell can provide dispersibility in both polar and nonpolar solvents. Additionally, various functional groups can be introduced onto the silica surface in order to conjugate the nanoparticles with other molecules or substrates. For example, the nanoparticles can be coated with cationic polyethyleneimine and strongly bind to the negatively charged bacterial cell surface. Peptides that recognize specific strains of bacteria can be conjugated to the nanoparticles and introduce targeting capability.

The nanoparticles of the invention may be prepared, for example, using a silver nanocrystal as a seed for the growth of the silica nanoparticles.

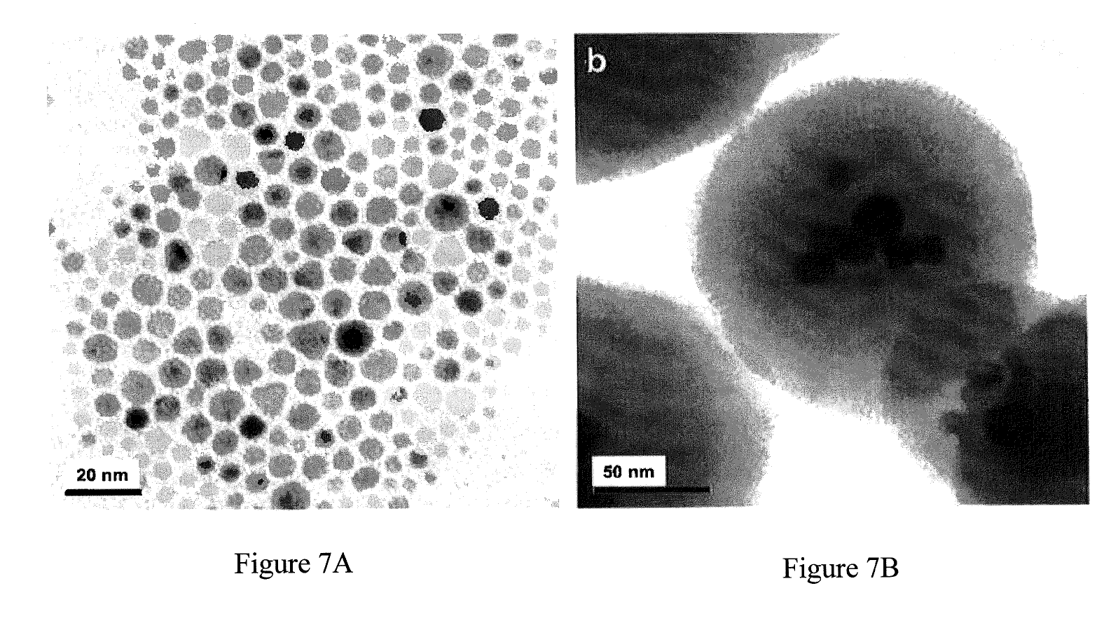

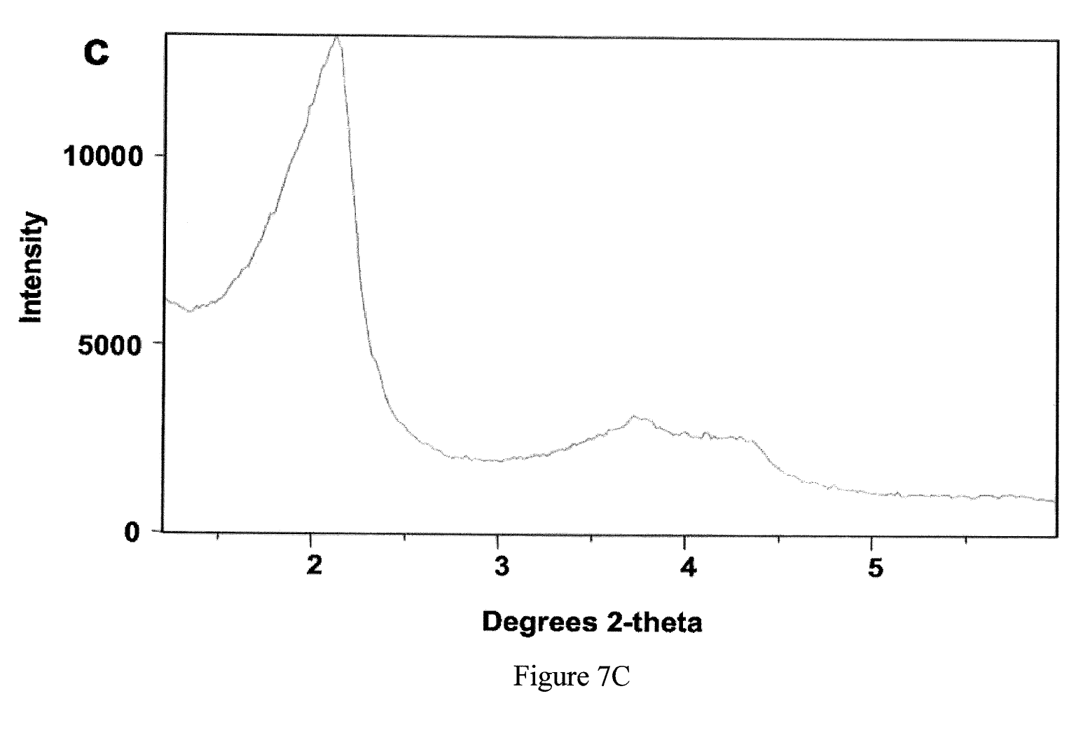

Hydrophobic silver nanocrystals synthesized through a modified non-hydrolytic process (Hiramatsu et al., Chem. Mater., 2004, vol. 16, p. 2509) may be used. For example, silver acetate may be reduced with oleylamine at high temperature to produce spherical oleylamine-capped silver nanocrystals that are less than 20 nm in diameter (FIG. 7). The nanocrystals are isolated by precipitation and washed, then redissolved in organic solvent (e.g. chloroform). The organic solution is then mixed with an aqueous mixture of cetyltrimethylammonium bromide (CTAB) surfactants, and the organic solvent evaporated to yield water-soluble silver nanocrystals (Kim et al., J. Am. Chem. Soc., 2006, vol. 128, p. 688).

The silica nanoparticles may be prepared, for example, by mixing the CTAB stabilized silver nanocrystals with tetraethylorthosilicate (TEOS) in basic aqueous solution (e.g. pH˜11). The solution is stirred at high temperature (e.g. 65-80° C.) to produce structured nanoparticles. An ion-exchange procedure of heating the nanoparticles in an ethanolic solution of ammonium nitriate may be used to remove the CTAB surfactants (Lang et al., Chem. Mater., 2004, vol. 16, p. 1961).

Silica nanoparticles having other pore sizes may be prepared, for example, by using different surfactants or swelling agents during the preparation of the silica nanoparticles.

The nanoparticles may then be treated with surface modifying compounds, such as trihydroxysilyl propyl methyl-phosphonate, trialkoxysilane compounds, or cationic polymers or proteins to prepare surface-modified nanoparticles.

Alternatively, surface modifying compounds, such as trialkoxysilane compounds, may be mixed with the silver nanocrystals and tetraethylorthosilicate to produce surface-modified nanoparticles directly.

As described previously, surface-modified nanoparticles bearing certain functional groups, such as amines, sulfhydryls, disulfides, expoxides, and/or carboxylic acids, may be further derivatized with proteins or light emitting compounds, using chemistry known in the art.

Since the nanoparticles are stable in an aqueous environment, they can be used to bind and eliminate pathogens present in solution. Additionally, the nanoparticles can also be used to functionalize fabrics, membranes, or other substrates (drop-in technology), which can incorporate the antimicrobial materials in composites.

Embodiments of the invention include an antimicrobial composition comprising a plurality of submicron structures described above. All of the previously described submicron structures or nanoparticles have a silver core. The silver core within the nanoparticle slowly dissolves to release antimicrobial silver ions.

The unmodified or surface-modified nanoparticles described above interact with various microbes. For example, nanoparticles coated with cationic poly(ethyleneimine) (PEI) bind strongly to negatively charged bacterial cell surfaces. Likewise, the surface of the nanoparticles may be modified by proteins designed to recognize a specific type of microbe, for instance a specific strain of bacteria. In this way, the nanoparticles can be targeted to a specific microbe. For example, nanoparticles with a surface modified by the Anthrax toxin receptor (ANTXR) protein may be used to recognize Bacillus anthracis specifically.

The unmodified or surface modified nanoparticles described above may also be suspended in liquid, for example for the preparation of antibacterial hand-washes, lotions, creams, ointments, cosmetics, toothpaste, mouthwashes, disinfectant sprays, or cleaning solutions. Charged modified and unmodified nanoparticles form stable suspensions in water. Nanoparticles bearing hydrophobic surface modifying groups are stable in organic solvents.

The antibacterial composition may also include a polymer or polymer blend. The modified or unmodified nanoparticles may be blended with polymers, providing an antimicrobial effect. The antibacterial polymer compositions may be used, for example, to coat surfaces of appliances such as washing machines or refrigerators to provide an antimicrobial property. Such coatings may also be useful for use in hospitals, to reduce the growth of bacteria, including highly drug resistant bacteria.

The modified or unmodified nanoparticles also adhere to fibers, such as cloth or paper, providing an antimicrobial material. Such materials may be used, for example in clothing, water filters, or air filters.

Other embodiments of the invention include methods of killing or inhibiting growth of a microbe by contacting the microbe with the submicron structures or nanoparticles described above. As used herein “inhibiting growth” or “inhibiting” means to reduce or inhibit replication of the microbe or reduce the growth rate of a colony or population of microbes. In some embodiments, the microbe is a bacteria, for example, Gram negative bacteria, or Gram positive bacteria.

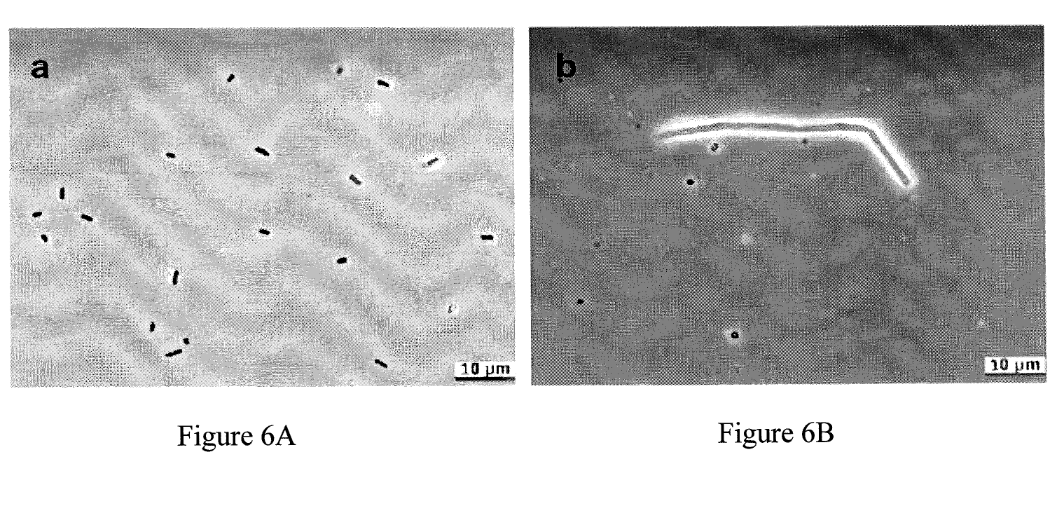

The nanoparticles have been shown to be effective at killing or inhibiting growth of bacteria, including Gram negative (e.g. E. coli) and Gram positive (Bacillus anthracis) bacteria. Without wishing to be bound by theory, one possible mechanism may involve nanoparticles associating closely with the external surface of the microbe, resulting in a high local concentrations of silver ions dissociating out of the nanoparticles, sufficient to kill or inhibit growth of the microbe. This explanation is supported by experiments showing fluorescent dye modified nanoparticles associating with the surface of bacteria, as shown in FIG. 6.

Surface modification of the nanoparticles may be used to enhance the interaction of nanoparticles with microbes, or to target the nanoparticles to specific microbes, as discussed previously.

Silver has been used to kill or inhibit growth of numerous microbial pathogens including bacteria, viruses, fungi, and microbial parasites. Any microbe that can be killed or inhibited by silver or ionic silver can be treated with the nanoparticles of the present invention. Ionic silver is used for literally hundreds of conditions, including eye and ear infections, nose, sinus and gum infections, acne, sore throats, colds and flu, candida, bladder and vaginal infections, cuts and burns, many skin conditions, bug bites, fighting nail and skin fungus, healing sunburn, alleviating diaper rash and bed sores, providing a soothing skin treatment after shaving, and use as a mouth rinse. Body odors are caused by bacteria in the perspiration, may be alleviated. Ionic silver is also used for treating ulcers, both in fighting the bacteria that can aggravate an ulcer and in repairing the damaged stomach lining. Ionic silver is used for many severe conditions as well, including, for example, tuberculosis, Epstein-Barr Virus, Lyme Disease, Legionnaires' Disease, bronchitis, chicken pox, and numerous others. There are actually few germ-related conditions, or conditions requiring the repair of tissue, for which ionic silver is not used, since many claim it is not only effective in killing most bacteria but also many if not most fungus and viruses. Some reports indicate that it is also effective against a number of parasites that might invade the body. Ionic silver is also reported by some researchers to be effective at treating cancer and HIV.

Specific examples of human pathogens include Streptococcus pyogenes (also known as Group A Strep; GAS; or flesh eating bacteria), Group B streptococcus, Staphylococcus aureus (including methicillin resistant Staphylococcus aureus or MRSA), Clostridium difficile, Pseudomonas aeruginosa, Klebsiella pneumoniae, and Mycobacteria tuberculosis. In addition, bacteria associated with food poisoning, including pathogenic E. coli, salmonella, shigella, Campylobacter jejuni and Clostridium perfringens, may be killed or inhibited by nanoparticles according to the invention. These organisms represent a significant impact to human health and additionally have a great economic impact. The effectiveness of nanoparticles according to the invention against E. coli are presented in the examples below.

As a matter of national security, potential biological weapons such as, Bacillus anthracis (etiological agent of anthrax), Francisella tularensis, Yersina pestis (etiological agent of plague); Clostridium botulinum and C. tetani, and Brucella, Burkholderia, and Coxiella species may also be killed or inhibited by nanoparticles of the invention. The effectiveness of the nanoparticles against B. anthracis are presented in the examples below.

Antibiotic-resistant microorganisms cause numerous problems and infections in various facilities. Although the antimicrobial activity of silver nanoparticles is well known and has proven effective against antibiotic-resistant strains, the materials are typically prone to aggregation and incompatible in a biological environment. By coating the silver nanocrystals with a porous silica shell, the materials allow for slow dissolution of the ions to induce the antimicrobial effect and prevent the aggregation issues.

From the foregoing description, it will be apparent that variations and modifications may be made to the invention described herein to adopt it to various usages and conditions. Such embodiments are also within the scope of the following claims.

The recitation of a listing of elements in any definition of a variable herein includes definitions of that variable as any single element or combination (or subcombination) of listed elements. The recitation of an embodiment herein includes that embodiment as any single embodiment or in combination with any other embodiments or portions thereof.

Terms listed in single tense also include multiple unless the context indicates otherwise.

The examples disclosed below are provided to illustrate the invention but not to limit its scope. Other variants of the invention will be readily apparent to one of ordinary skill in the art and are encompassed by the appended claims. All publications, databases, and patents cited herein are hereby incorporated by reference for all purposes.

Methods for preparing, characterizing and using the compounds of this invention are illustrated in the following Examples. Starting materials are made according to procedures known in the art or as illustrated herein. The following examples are provided so that the invention might be more fully understood. These examples are illustrative only and should not be construed as limiting the invention in any way.

### EXAMPLES

In summary, the synthesis of silver nanocrystals encapsulated in mesoporous silica nanoparticles with a yolk-shell structure are described, and their antimicrobial effect in both liquid media and LB-agar plates are demonstrated. The methods used to synthesize the structured nanoparticles are versatile enough to incorporate multiple types of inorganic nanocrystals and the silicate exterior allows further surface modification through either covalent or electrostatic interactions. These silver-containing nanoparticles may be used as a promising alternative to the current technologies involving the use of silver nanoparticles and silver-doped materials as antimicrobial coatings (Kumar et al., Nat. Mater., 2004, vol. 7 p. 485; Loher et al., Small, 2008, vol. 4, p. 824) and colloidal suspensions (Lok et al., J Proteome Res., 2006, vol. 5, p. 916).

### Example 1

### Synthesis of Silver Nanocrystals

The hydrophobic silver nanocrystals used as the seed template were synthesized through a modified non-hydrolytic process. (Hiramatsu et al., Chem. Mater. 2004, vol. 16, p. 2509) In comparison to the aqueous phase synthesis which requires large amounts of solvents (Pal et al., Appl. Environ. Microbiol, 2007, vol. 73, p. 1712), the non-hydrolytic method is more suitable since it requires inexpensive reagents and yields larger quantities of products. The reduction of silver acetate with oleylamine at high temperature resulted in spherical oleylamine-capped silver nanocrystals that are less than 20 nm in diameter (FIG. 7). Silver acetate (50 mg, Sigma, 99%) was dissolved in oleylamine (2.5 mL, Aldrich, 70%) and quickly added into a boiling solution of toluene (50 mL). The mixture was refluxed and stirred vigorously for 12 h under nitrogen. After removing the toluene using rotary evaporation, methanol was added into the solution to precipitate the silver nanocrystals. After collecting the nanocrystals using centrifugation, the materials were washed with methanol and dried under vacuum.

After the nanocrystals were isolated from the solution by precipitation and washed to remove excess starting reagents, they were redissolved in chloroform. The nanocrystal solution was mixed with an aqueous mixture of cetyltrimethylammonium bromide

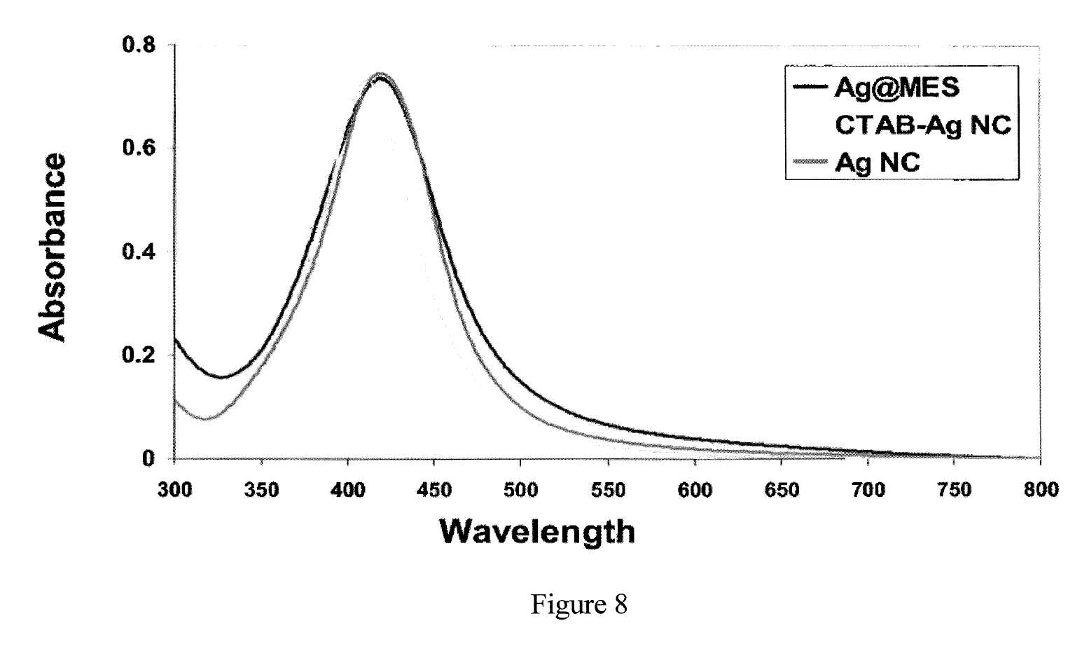

(CTAB) surfactants, and the chloroform was evaporated to yield water-soluble silver nanocrystals (Kim et al., J. Am. Chem. Soc., 2006, vol. 128, p. 688). As shown in the UV-Vis extinction spectrum (FIG. 8), the surface plasmon absorption band of water-soluble CTAB-stabilized silver nanocrystals was blue-shifted compared to the as-synthesized nanocrystals due to the change in the dielectric medium (Mulvaney, Langmuir, 1996, vol. 12, p. 788). Unlike iron oxide and gold nanocrystals, the oleylamine-capped silver nanocrystals are not stable when mixed with the CTAB surfactants. Although the aqueous nanocrystal solution becomes clear after the removal of the organic solvent, the materials must be coated with mesoporous silica quickly since they tend to precipitate after 30 min at room temperature.

### Example 2

### Synthesis of Silver Encapsulated Mesoporous Silica Nanoparticles (Ag@MESs)

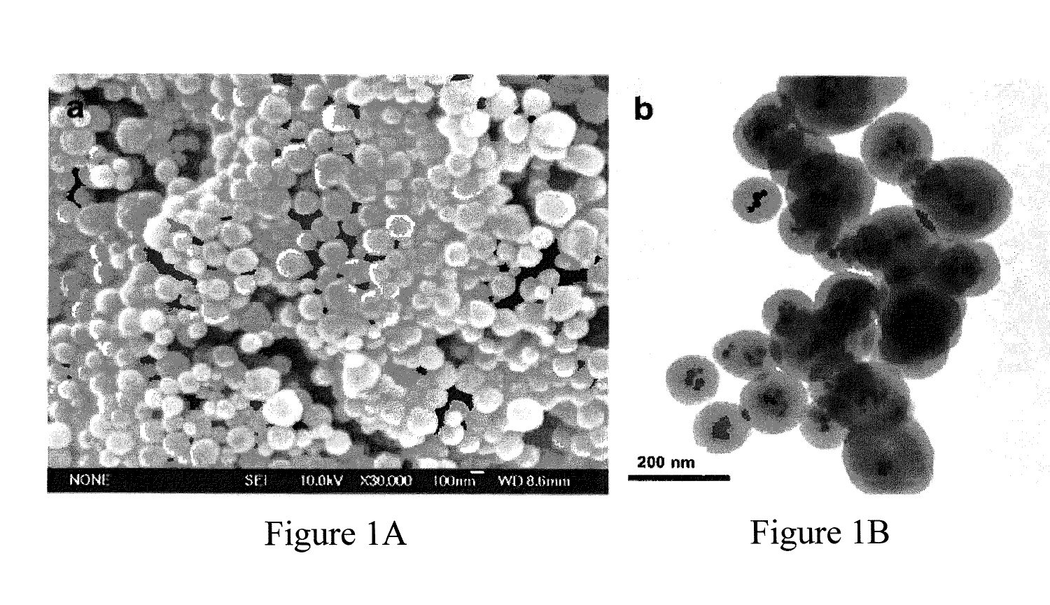

The Ag@MESs were prepared by mixing CTAB-stabilized silver nanocrystals with the silica source, tetraethylorthosilicate (TEOS), in a basic aqueous solution (˜pH 11). The electrostatic interaction between hydrolyzed TEOS molecules, CTAB-stabilized nanocrystals, and free surfactant micelles quickly led to the formation of mesostructured particles (Fan et al., Science, 2004, vol. 304, p. 567). Since the particle morphology is highly dependent on the reaction condition, the solution is stirred vigorously and heated at high temperature (−80° C.) to form the yolk-shell structured nanoparticles. The electron microscope images in FIG. 1 shows the Ag@MESs in which multiple silver nanocrystals are embedded at the center of the spherical mesoporous silica structure. An ion-exchange procedure of heating the particles in an ethanolic solution of ammonium nitrate was used to remove the toxic CTAB surfactants (Lang et al., Chem. Mater., 2004, vol. 16, p. 1961). Using this solvent extraction process, the surfactants were removed from the pores without damaging the mesostructure or morphology of the particles as confirmed by transmission electron microscopy and X-ray diffraction analysis (FIG. 7). Furthermore, the plasmon absorption band of the surfactant-removed Ag@MESs remains intact and is similar to that of the original oleylamine-capped silver nanocrystals (FIG. 8). When the as-synthesized Ag@MESs were instead heated in acidic alcohol solution for the surfactant removal process, the silver nanocrystals would dissolve in less than an hour, thus confirming the accessibility of the mesostructured network in allowing the oxidation and dissolution of the core materials.

In a 25 mL flask, deionized water (8.6 mL) and sodium hydroxide (70 μL, 2 M) was stirred vigorously and heated to 80° C. The silver nanocrystals were dissolved in chloroform at ˜20 mg/mL concentration. In a separate container, the silver nanocrystals (100 μL) was then mixed with a solution of cetyltrimethylammonium bromide (20 mg, CTAB, Aldrich, 95%) dissolved in water (1 mL), and sonicated thoroughly. After evaporating the chloroform, the CTAB-silver nanocrystals were centrifuged for 1 min at 14000 RPM to remove precipitates or aggregates and the supernatant was added into the heated aqueous solution. After approximately 5 min, tetraethylorthosilicate (100 μL, TEOS, Sigma, 98%) was slowly added into the solution. Optionally, dye molecules such as Rhodamine B isothiocyanate (RITC, Sigma) can be incorporated on the materials by first dissolving RITC (1 mg) in absolute ethanol (0.6 mL) and allowing it to react with aminopropyltriethoxysilane (2.4 μL, Aldrich, 99%) for 2 h. The ethanolic RITC-silane solution (120 μL) was then mixed with TEOS (100 μL) before slowly adding them to the CTAB-nanocrystals solution. The mixture was stirred for an additional 2 h, collected by centrifugation, and washed with ethanol. To remove the surfactants from the mesopores, the materials were dispersed in a solution of ethanol (12 mL, 95%) and ammonium nitrate (32 mg), and heated for 40 min. The process was repeated and the nanoparticles were thoroughly washed with ethanol and deionized water.

### Example 3

### Synthesis of Superparamagnetic Nanoparticles

Superparamagnetic iron oxide nanocrystals were synthesized by following the modified procedure described by Park et al. (Nat. Mater. 2004, vol. 3, p. 891). The iron oxide nanocrystals were synthesized by the thermal decomposition of iron-oleate complex in nonpolar solution. 2.2 g iron (III) chloride hexahydrate and 7.4 g sodium oleate were dissolved in a mixture of 16.3 mL absolute ethanol and 12.2 mL water, and mixed with 28.5 mL hexane; the solution was refluxed for 4 h. The mixture was then washed with water several times in a separatory funnel and the hexane was removed from the mixture by using rotary evaporation. The synthesized iron-oleate complex was then dried under vacuum overnight. 1 g of iron-oleate complex was dissolved in a solution of 177.3 μL oleic acid and 7.1 mL octadecene. The mixture was placed under vacuum and heated at 80° C. for 30 min. It was then stirred vigorously under inert atmosphere and heated to 320° C. at a rate of 3° C./minute and kept at that temperature for 30 min. After the mixture has cooled to room temperature, hexane was added and the nanoparticles were precipitated by adding an excess of ethanol. The nanoparticles were separated from the solution by centrifugation. The nanoparticles were then washed twice in a solution of 1:5 hexane-ethanol using centrifugation and dried under vacuum.

The iron oxide nanocrystals were made water soluble by using similar procedure described for the silver nanocrystals. To synthesize mesoporous silica nanoparticles embedded with mixed iron oxide and silver nanocrystals, similar procedures were also done with some modifications. CTAB-iron oxide nanocrystals (0.5 mL, 2 mg/mL) and of CTAB-silver nanocrystals (0.5 mL, 2 mg/mL) were instead added into the heated aqueous basic solution. The reactions were usually performed at 65-70° C. rather than 80° C. in order to form the yolk-shell structure.

### Example 4

### Polyethylene Imine Coating

The nanoparticles (5 mg) were dispersed in a solution of polyethyleneimine (2.5 mg, PEI, Mₙ 1200, Aldrich) and absolute ethanol (1 mL). After the mixture was sonicated and stirred for 30 min, the PEI-coated particles were washed with ethanol and deionized water.

### Bacteria Experiments

E. coli BL21 DE3 was purchased from Invitrogen Corporation. Bacillus anthracis strain BH450 was provided by Dr. Stephen H. Leppla at the National Institutes of Health. Luria-Bertani Lennox media (VWR) was used in growing and maintaining the bacterial cultures.

### Example 5

### Liquid Media

For the growth curve experiments, a starter culture of each strain was inoculated with fresh colonies and incubated for 14 h overnight in LB Lennox media. Bacterial growth rates were determined by measuring the optical density at 600 nm via spectrophotometer (Eppendorf BioPhotometer). Fresh media (25 mL) was inoculated with the starter culture and grown to an OD₆₀₀ of 0.1 at 37° C. with continuous agitation at 250 rpm. Various concentrations of nanoparticles were then added to the culture and the turbidity measurements were taken over a time course.

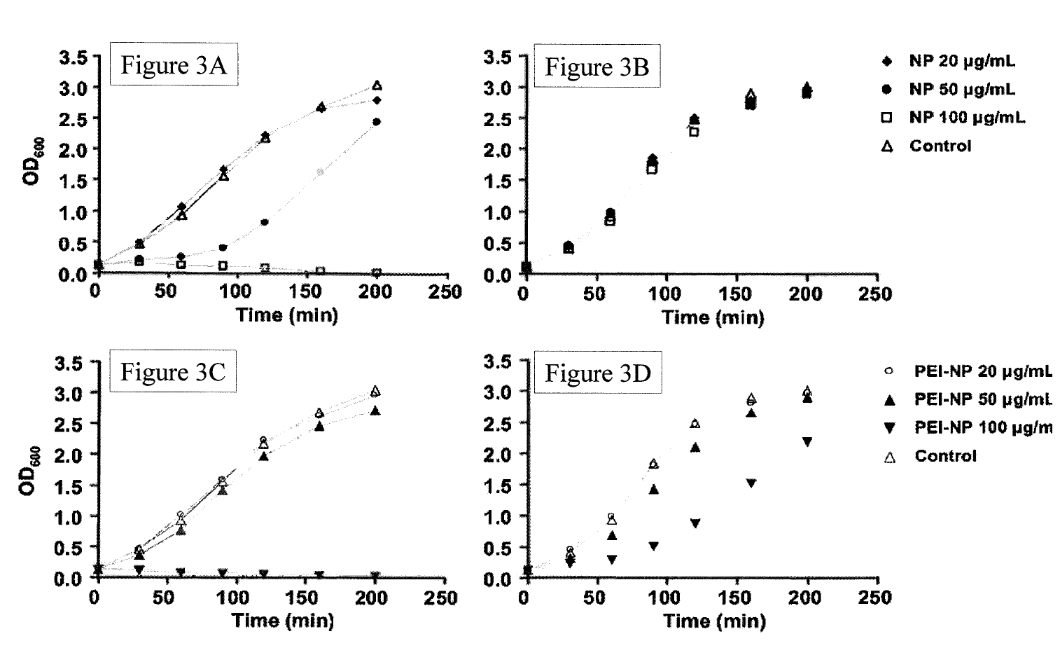

The effect of encapsulated silver nanoparticles on the bacteria growth kinetics in liquid media was studied (FIG. 3). The bacterial growth was monitored by measuring the optical density at 600 nm (OD₆₀₀) based on the turbidity of the cell suspension (Thiel et al., Small, 2007, vol. 3, p. 799). For these experiments, bacteria were grown to an OD₆₀₀=0.1, and then mixed with various concentrations of Ag@MESs. Since the Ag@MESs can interfere with the optical density reading at that wavelength, the appropriate concentration of particles was added into the media and used as the background measurement. The nanoparticles were able to slow the growth of B. anthracis at 50 μg/mL and completely inhibited its growth at 100 μg/mL. On the contrary, the Ag@MESs did not have noticeable effect on E. coli growth at all tested concentrations as the curve was similar to that of the control sample, which was not supplemented with nanoparticles.

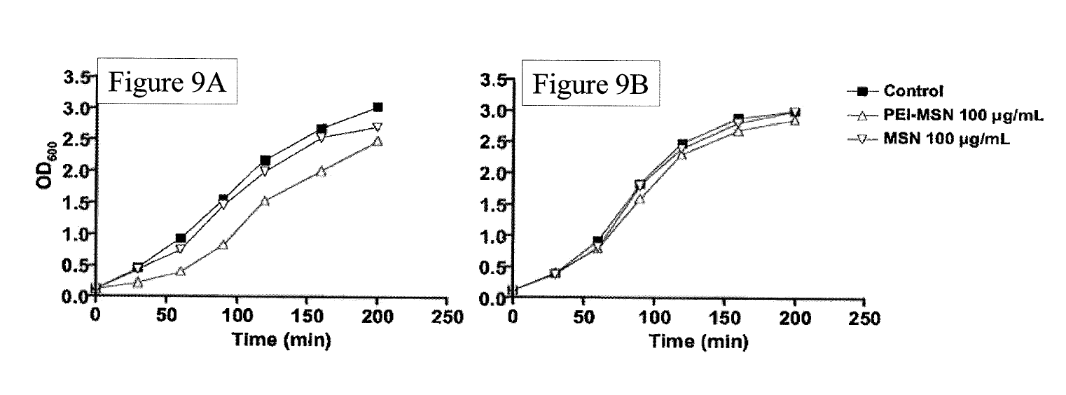

To further investigate the effect of nanoparticle surface on the cell growth, Ag@MESs were coated with low molecular weight polyethyleneimine (PEI) (Example 4) through electrostatic interactions to create a positively charged surface (Fuller et al., Biomaterials, 2008, vol. 29, p. 1526). The cationic surface modification were believed to increase the particle association with the negatively charged bacterial surface. The anionic Ag@MESs and cationic PEI-coated Ag@MESs exhibited zeta potential values of −22 mV and 82 mV, respectively, when measured in Milli-Q deionized water (pH 6.5). It was observed that cationic PEI-coated Ag@MESs were more effective in slowing the growth of E. coli compared to anionic particles (FIG. 3). This result is consistent with another report which demonstrates that cationic gold particles have greater affinity towards the negatively charged lipopolysaccharide layer that coats E. coli and other Gram-negative bacteria (Phillips et al., Angew. Chem., Int. Ed., 2008, vol. 47, p. 2590). The surface modification, however, had a less noticeable effect on B. anthracis. At 100 μg/mL, both types of particles completely inhibited bacterial growth, but at 50 μg/mL, the delay in bacterial growth was observed only for negatively charged Ag@MESs. Because the bacteria were cultured in optimal growth conditions, the cells that were minimally affected by the nanoparticles continued to multiply as observed in the slower growth kinetics. Mesoporous silica nanoparticles (Cai et al., Chem. Mater., 2001, vol. 13, p. 258) without encapsulated silver nanocrystals were also tested to confirm that the growth inhibition observed was caused by the silver and not the silicate materials. FIG. 9 shows that the growth curves of the bacteria that had been treated with the mesoporous silica nanoparticles (with and without PEI coating, 100 μg/mL concentration) were similar to those of the control samples.

### Example 6

### LB-Agar Plates

The nanoparticles were mixed with molten LB-agar at varying final concentrations (20, 50, and 100 μg/mL). Serial dilution (1/10⁴) of late log phase bacteria (OD₆₀₀=2.0) were then plated onto solidified silver nanoparticle agar plates and incubated at 37° C. for 24 h.

### Results

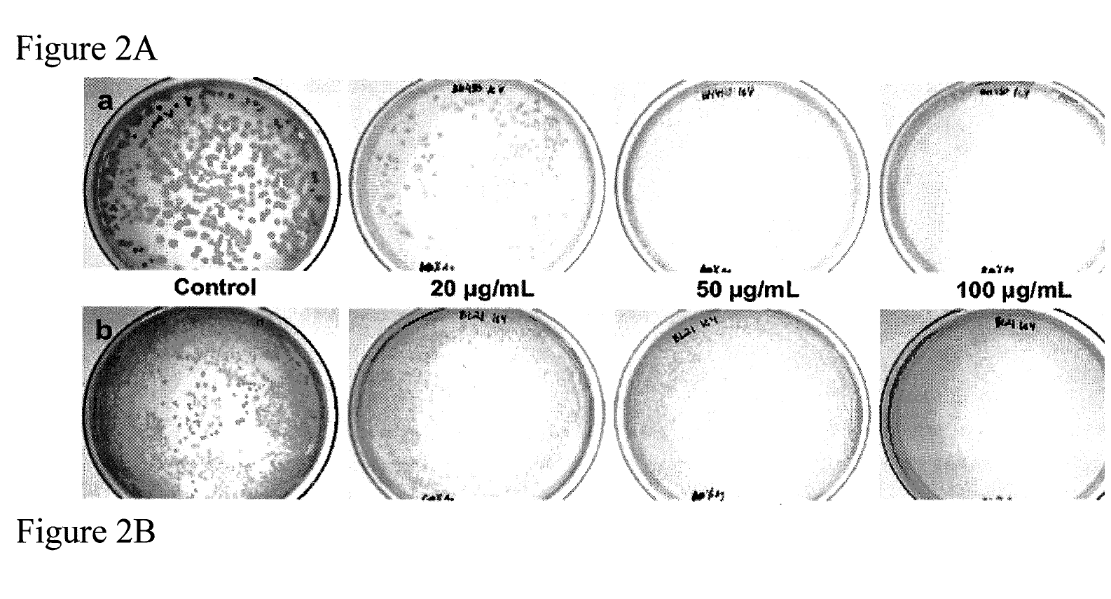

The antimicrobial efficacy of the materials was investigated by supplementing LB-agar media with an aqueous suspension of Ag@MESs at various concentrations. The nanoparticles were added to the molten LB-agar solution and the mixture was allowed to solidify at room temperature. The suspension of bacteria was then spread onto the Ag@MES-containing LB-agar plates and incubated overnight in the dark. The presence of Ag@MESs in the LB-agar plates was able to inhibit the formation of colonies for both types of bacteria (FIG. 2). The effect was more noticeable for the B. anthracis compared to the E. coli as the nanoparticles were able to substantially reduce the number of colonies at a final Ag@MES concentration of 20 μg/mL. The formation of colonies for both strains was fully inhibited when the LB-agar plates contained 100 μg/mL of the particles.

### Discussion

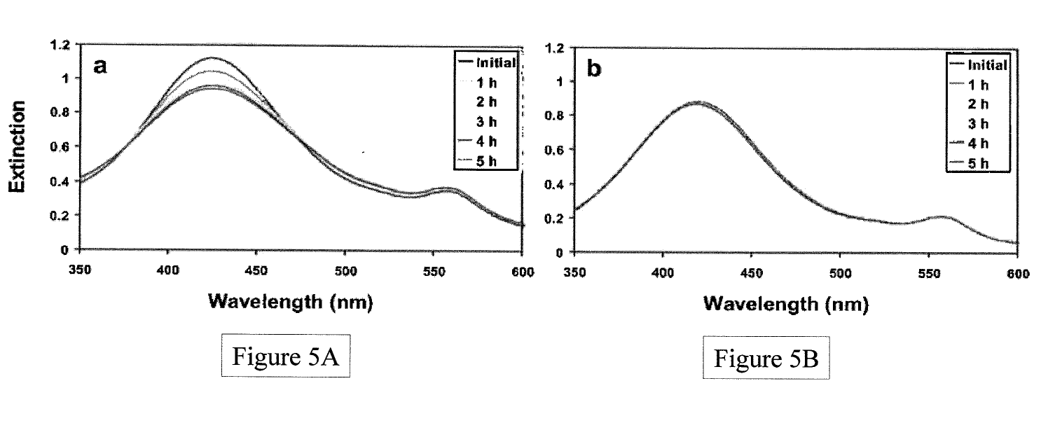

The decrease in bacterial growth that was observed both in liquid media and LB-agar plates was likely caused by the oxidative chelation process of the bulk nanocrystals into silver ions (Sondi et al., J Colloid Interface Sci, 2004, vol. 275, p. 177; Ung et al., Langmuir, 1998, vol. 14, p. 3740). By measuring the plasmon peak of the silver nanocrystals, the dissolution of the materials could be observed (Ung et al., Langmuir, 1998, vol. 14, p. 3740). The absorption of the RITC-labeled Ag@MES suspension was monitored over a time period in LB Lennox culture medium and also in Milli-Q deionized water (FIG. 5). A large decrease in the plasmon band at 425 nm, especially within the first hour, corresponds to the oxidation of silver nanocrystals in the culture medium. When the nanoparticles were suspended in deionized water, however, there was only a minimal decrease in the plasmon peak over the time period. For comparison, the Rhodamine B absorption peak at 560 nm remained relatively similar, confirming that the decrease in plasmon band was not caused by a change in the concentration of Ag@MESs or the sedimentation of the particles. The culture medium contains various salts and peptides that can contribute to the oxidation of the materials into silver ions and lead to the growth inhibition observed for both liquid medium and LB-agar plates (Shrivastava et al., Nanotechnology, 2007, vol. 18, p. 225103; Ho et al., Adv. Mater, 2004, vol. 16, p. 957). Since the precursors to make LB-agar also contain salts and peptides, this likely resulted in the release and even distribution of bactericidal silver ions in the LB-agar plates and prevented the bacteria from forming colonies. It remains unclear why the Ag@MESs were able to inhibit E. coli colony formation in the LB-agar plates, but were only able to delay the bacterial growth in culture media, although a similar occurrence has been reported (Sondi et al., J Colloid Interface Sci, 2004, vol. 275, p. 177).

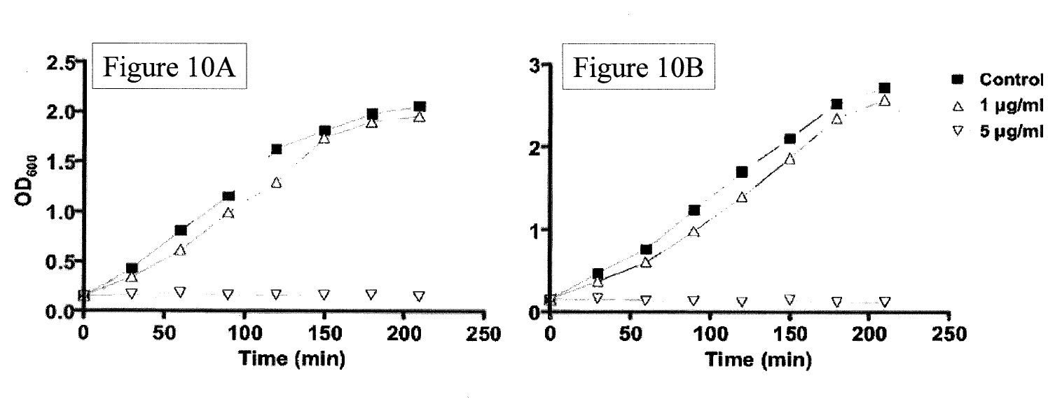

Additional studies were done to confirm that silver ions do inhibit the bacterial growth and that neither type of bacteria is more susceptible to silver ions (FIG. 10). Silver nitrate was used instead of silver acetate (precursor of the silver nanocrystals) for the source of silver ions due to its water solubility. At 5 μg/mL concentration, the silver nitrate completely inhibited the growth of both B. anthracis and E. coli, whereas at 1 μg/mL, the growth of both types of bacteria was unaffected and appeared similar to the control samples.

### Example 7

### Fluorescence Microscopy

Late log phase bacteria (OD₆₀₀⁼2.0) were incubated with RITC-labeled nanoparticles at a final concentration of 10 μg/mL in phosphate buffered saline (pH=7.3) for 30 min. The bacteria were then washed twice and resuspended in PBS. A sample was placed on a cover slip and imaged using Zeiss Axio Imager Z1 at 594 nm.

### Results

Studies of the interaction of mesoporous silicate nanoparticles with bacteria are rare. The nanoparticles were modified with dye molecules to enable studies of possible interaction using fluorescence microscopy. Rhodamine B isothiocyanate (RITC) was reacted with aminopropyltriethoxysilane and mixed with the silica precursor tetraethylorthosilicate (TEOS) to functionalize the interior pores and surface of the particles with the dye molecules without disrupting the mesostructure (Lu et al., Small, 2007, vol. 3, p. 1341; Slowing et al., J. Am, Chem. Soc. 2006, vol. 128, p. 14792) Two types of bacteria were used: Bacillus anthracis BH450 (B. anthracis) as the Gram-positive model and Escherichia coli BL21 DE3 (E, coli) as the Gram-negative model. Upon mixing the nanoparticles with bacteria, red fluorescence of the nanoparticles was found to overlap with B. anthracis, but not with E. coli, suggesting that the particle adherence to the bacteria may depend on the bacterial strain and surface characteristic of the nanoparticles (FIG. 6).

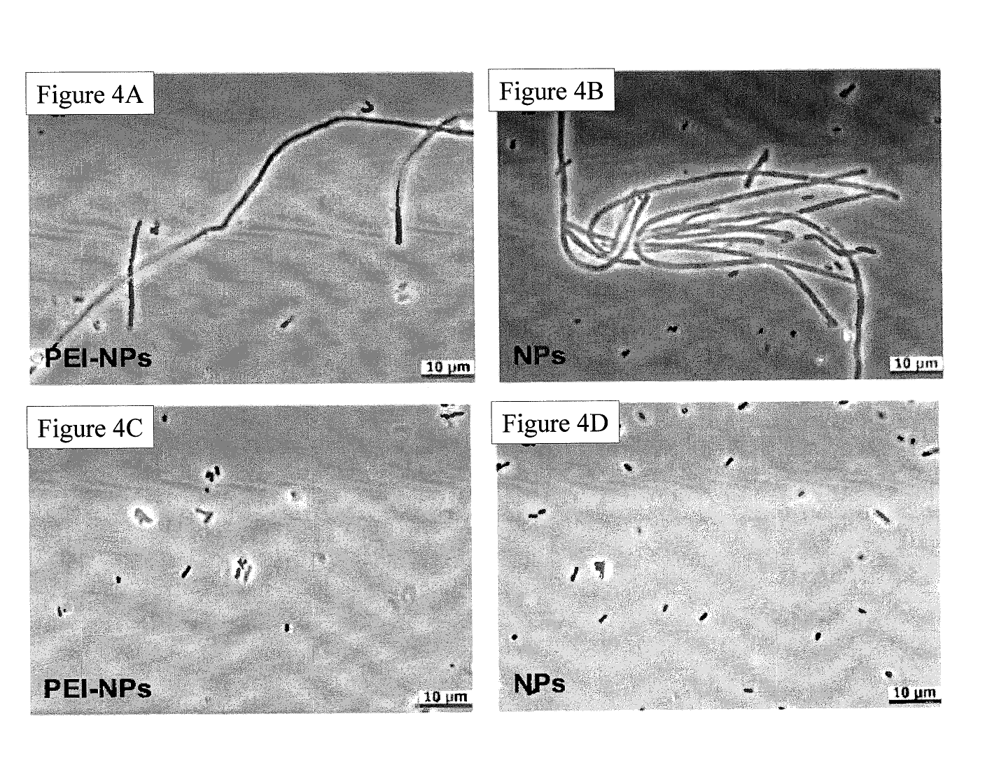

The interaction between the encapsulated silver particles and the bacteria was observed using fluorescence microscopy. Ag@MESs were fluorescently labeled with RITC using similar methods for labeling the mesoporous silica nanoparticles. The association of the Ag@MESs with the bacteria depended on the surface characteristic of the nanoparticles and correlated with the cytotoxicity. For Gram-negative E. coli, it was observed that the positively charged PEI-coated Ag@MESs have greater affinity towards the bacteria as most of the red fluorescence from the nanoparticles was prominent on the bacterial surface (FIG. 4). On the other hand, the negatively charged Ag@MESs were dispersed throughout the microscope slides rather than on the bacterial surface similar to the results obtained with mesoporous silica nanoparticles (FIG. 6). This result correlates with the viability assay experiments in which the PEI-coated particles show a noticeable inhibitory effect on the bacteria growth curve. In the case of Gram-positive B. anthracis, both types of particles were observed on the bacterial surface. A large amount of red fluorescence was associated with B. anthracis, caused by the strong interaction of particles with the bacterial surface (FIG. 4). Although the PEI-coated Ag@MESs did associate with bacilli, the negatively charged particles showed a slightly increased association to bacilli in contrast to the result observed with the E. coli experiments. While this result is unexpected for many Gram-positive bacteria that display negatively-charged teichoic acid on the peptidoglycan layer (Berry et al., Angew. Chem., Int. Ed., 2005, vol. 44, p. 6668), the B. antharacis strain used produces a proteinacious, crystalline S-layer that surrounds the bacillus and likely governs interactions with nanoparticles (Mignot et al., Environ. Microbiol., 2001, vol. 3, p. 493). This observation also correlates with the viability assay experiments in which the Ag@MESs were able to slow the growth of B. anthracis at 50 μg/mL as opposed to the PEI-coated samples. Fluorescence microscopy and cell viability assays thus confirm the importance of particle association with the bacterial surface. The Ag@MESs are able to affect bacterial growth much more effectively when they bind and are in close proximity to the bacterial surface.

### Example 8

### Superparamagnetic Silica Nanoparticles

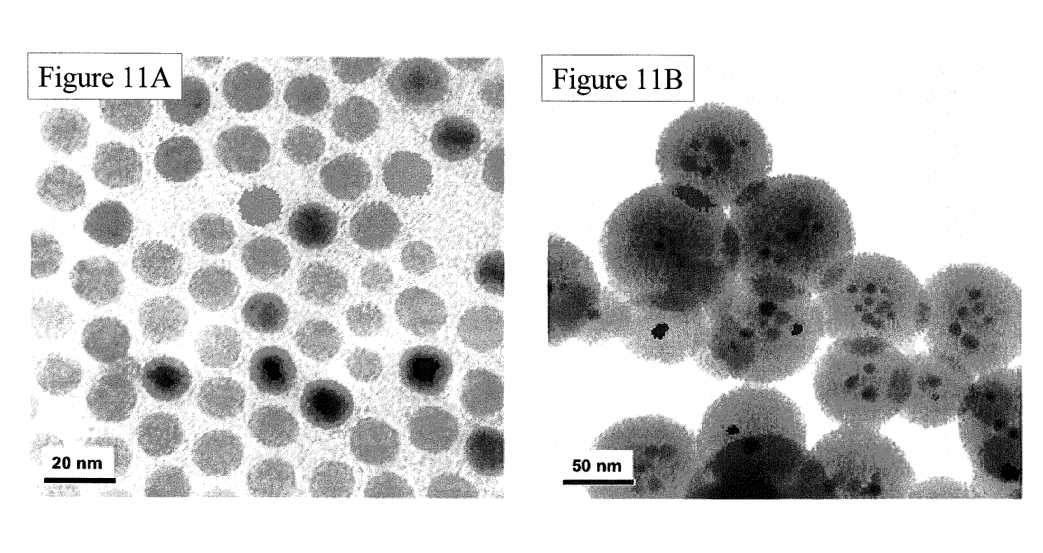

The versatility of using mesoporous silica nanoparticles as a delivery vehicle for the slow release of bactericidal silver ions can be demonstrated further by encapsulating both iron oxide and silver nanocrystals within the materials. By rendering the superparamagnetic iron oxides water-soluble with CTAB surfactants (Kim et al., J. Am. Chem. Soc., 2006, vol. 128, p. 688) and mixing both of the nanocrystal-surfactant solutions, mesoporous silica nanoparticles with iron oxide and silver nanocrystals incorporated at the core of the particles were produced (FIG. 11).

To synthesize mesoporous silica nanoparticles embedded with mixed iron oxide and silver nanocrystals, similar procedures were also done with some modifications. 0.5 mL of CTAB-iron oxide nanocrystals (2 mg/mL) and 0.5 mL of CTAB-silver nanocrystals (2 mg/mL) were instead added into the heated aqueous basic solution containing 8.6 mL water and 70 μL sodium hydroxide (2 M). The reactions were usually performed at 65-70° C. rather than 80° C. in order to form the yolk-shell structure. After approximately 5 min, 100 μL tetraethylorthosilicate (TEOS) was slowly added into the solution. Optionally, dye molecules such as Rhodamine β isothiocyanate (RITC) can be incorporated on the materials by first dissolving 1 mg of RITC in 0.6 mL absolute ethanol and allowing it to react with 2.4 μL aminopropyltriethoxysilane for 2 h. 120 μL of the ethanolic RITC-silane solution was then mixed with 100 μL TEOS before slowly adding them to the CTAB-nanocrystals solution. The mixture was stirred for an additional 2 h, collected by centrifugation, and washed with ethanol. To remove the surfactants from the mesopores, the materials were dispersed in a solution of 12 mL ethanol (95%) and 32 mg ammonium nitrate, and heated for 40 min. The process was repeated and the nanoparticles were thoroughly washed with ethanol and deionized water.

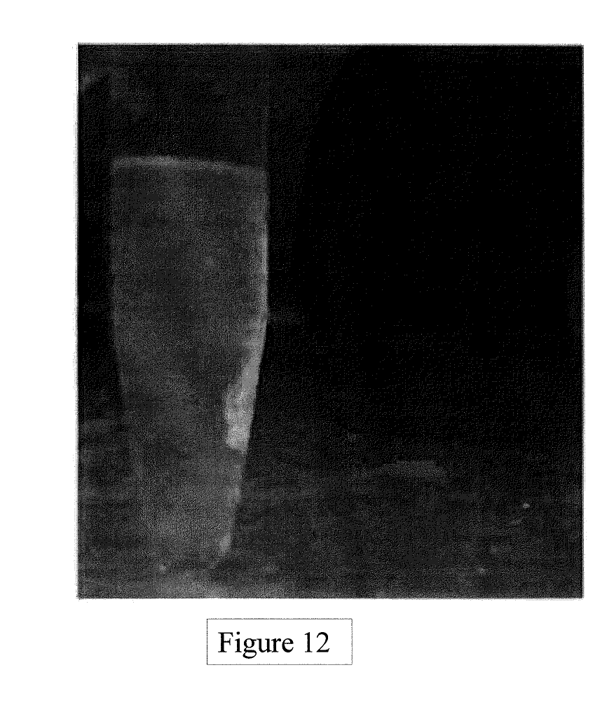

The binding of superparamagnetic PEI-coated particles to E. coli (stained with fluorescent DNA-binding dye Hoechst 33342) enabled the bacteria to be collected by a neodymium magnet (FIG. 12). Following the works in which silica nanoparticles were used to specifically bind to pathogenic bacteria through antibody recognition (Wang et al., Bioconjugate Chem., 2007, vol. 18, p. 297), it is believed that these magnetic and bactericidal particles can target specific strains of bacteria by conjugating antibodies to the surface. These targeted bacteria can potentially be separated from the non-targeted cells using an external magnetic field and/or eliminated by the slow release of silver ions (Chen et al., Small 2008, 4, 485).

### CONCLUSION

The synthesis of silver nanocrystals encapsulated in mesoporous silica nanoparticles (Ag@MESs) with a yolk-shell structure are described and the antimicrobial efficacy of the materials against both Gram-positive and Gram-negative bacteria were tested. Silver nanocrystals were used as the seed for the growth of silica nanoparticles as well as the source of antimicrobial silver ions. The porous silica shell made the hydrophobic silver nanocrystals compatible in aqueous solution and protected the active materials from aggregation. Due to the accessibility and porous network of the protective silica layer, the embedded nanocrystals are still able to be slowly oxidized into silver ions. Additionally, the silica component provided the durable support for surface modification with polyelectrolytes and silanes to affect the binding of the particles to the bacterial surface.

The embodiments illustrated and discussed in this specification are intended only to teach those skilled in the art the best way known to the inventors to make and use the invention. Figures are not drawn to scale. In describing embodiments of the invention, specific terminology is employed for the sake of clarity. However, the invention is not intended to be limited to the specific terminology so selected. The above-described embodiments of the invention may be modified or varied, without departing from the invention, as appreciated by those skilled in the art in light of the above teachings. It is therefore to be understood that, within the scope of the claims and their equivalents, the invention may be practiced otherwise than as specifically described.

1. A submicron structure, comprising:
a silver core; and
a silica body formed around said silver core, said silica body defining a plurality of pores and an outer surface between pore openings of said plurality of pores,
wherein said submicron structure has a maximum dimension less than one micron.

2. The submicron structure of claim 1, wherein the silica body is mesoporous.

3. The submicron structure of claim 1, wherein the pores are substantially cylindrical pores having an ensemble average diameter between about 1 nm and about 10 nm.

4. The submicron structure of claim 1, wherein the silica body is substantially spherical having a diameter between about 50 nm and about 1000 nm.

5. The submicron structure of claim 1, wherein the silica body is substantially spherical having a diameter between about 100 nm and about 500 nm.

6. The submicron structure of claim 1, wherein the silver core is a silver nanocrystal core with a maximum dimension less than about 50 nm.

7. The submicron structure of claim 6, wherein the silver nanocrystal has a maximum dimension less than about 20 nm.

8. The submicron structure of claim 1, further comprising a stopper assembly attached to said silica body, said stopper assembly comprising a blocking unit arranged proximate at least one said pore and having a structure suitable to substantially prevent material from being released while said blocking unit is arranged in a blocking configuration,
wherein said stopper assembly is responsive to the presence of a predetermined stimulus such that said blocking unit is released in the presence of said predetermined stimulus to allow said material to be released, and
wherein said predetermined stimulus is a predetermined catalytic activity that is suitable to at least one of cleave, hydrolyze, oxidize, or reduce a portion of said stopper assembly.

9. The submicron structure of claim 1, further comprising an impeller attached to said silica body.

10. The submicron structure of claim 1, further comprising a valve assembly attached to said silica body.

11. The submicron structure of claim 1, further comprising a surface modification.

12. The submicron structure of claim 11, wherein the surface modification comprises a plurality of anionic or electrostatic molecules attached to an outer surface of said silica body,
wherein said anionic or electrostatic molecules provide hydrophilicity or aqueous dispersability to said nanodevice and are suitable to provide repulsion between other similar submicron structures.

13. A submicron structure according to claim 12, wherein said plurality of anionic molecules comprise a phosphonate moiety.

14. A submicron structure according to claim 13, wherein said plurality of anionic molecules are trihydroxysilylpropyl methylphosphonate.

15. A submicron structure according to claim 11, wherein said surface modification comprises a functional group covalently bonded to the surface.

16. A submicron structure according to claim 15, wherein said functional group is an amine, sulfhydryl, disulfide, halide, carboxylic acid, epoxide, azide, alkyne, or hydrophobic moiety.

17. The submicron structure according to claim 16, wherein said functional group is covalently bonded to the surface via a C₁-C₁₂ alkyl linker.

18. The submicron structure according to claim 16, wherein the surface modification is further covalently bonded to a light-emitting molecule.

19. The submicron structure according to claim 16, further comprising a peptide, protein, oligonucleotide, sugar, oligosaccharide, or polysaccharide covalently or electrostatically bonded to said surface modification.

20. The submicron structure according to claim 19, wherein said peptide, protein, oligonucleotide, sugar, oligosaccharide, or polysaccharide is covalently bonded to said surface modification via a linker.

21. The submicron structure according to claim 20 wherein the peptide or protein is a targeting protein or antibody.

22. The submicron structure of claim 11, wherein said surface modification is electrostatically bonded to the surface.

23. The submicron structure of claim 22, wherein said surface modification is a polymer, protein, peptide, nucleic acid, sugar, oligosaccharide, polysaccharide or combinations thereof.

24. The submicron structure of claim 23, wherein said surface modification is a cationic polymer.

25. The submicron structure of claim 23, wherein said surface modification is a protein.

26. The submicron structure of claim 1, further comprising a second core structure within said silica body.

27. The submicron structure of claim 26, wherein said second core structure is a superparamagnetic nanocrystal.

28. The submicron structure of claim 27, wherein the superparamagnetic nanocrystal is an iron oxide nanocrystal.

29. The submicron structure of claim 26, wherein said second core structure is a gold nanocrystal.

30. An antimicrobial composition comprising a plurality of submicron structures according to claim 1.

31. The antimicrobial composition according to claim 30, further comprising a liquid, fiber, or polymer material.

32. The antimicrobial composition of claim 31 comprising a fiber material selected from the group consisting of cloth or paper.

33. A method of killing or inhibiting growth of a microbe comprising contacting said microbe with a submicron structure according to claim 1.

34. The method of claim 33, wherein the microbe is a bacteria.

35. The method of claim 34, wherein the bacteria is a Gram positive bacteria.

36. The method of claim 34, wherein the bacteria is a Gram negative bacteria.
